(19) United States
(12) Patent Application Publication
(10) Pub. No.: US 2026/0314592 A1
(43) Pub. Date: Oct. 15, 2026

(54) SPECIMEN CONTAINER FOR REPRODUCTIVE MEDICINE

(71) Applicant: Ta-Chin LIN, Tainan City (TW)

(72) Inventor: Ta-Chin LIN, Tainan City (TW)

(21) Appl. No.: 19/461,182

(22) Filed: Jan. 27, 2026

Related U.S. Application Data
(63) Continuation-in-part of application No. PCT/CN2023/109867, filed on Jul. 28, 2023.

(51) Int. Cl. A01N 1/146

(52) U.S. Cl. CPC A01N 1/146

(57) ABSTRACT

A specimen container for reproductive medicine includes a container body. The container body includes a base wall, a surrounding wall extending upwardly from the base wall to define a specimen receiving space, and a protrusion projecting upwardly from the base wall and formed in the specimen receiving space. The specimen receiving space has an outer surrounding region which is bordered between the protrusion and an inner peripheral surface of the surrounding wall. The structure of the container body is suitable for selecting and processing semen, ova, embryos or cell clusters. It can facilitate the selection or processing and can bring to standardization of the procedures, which therefore reduces damage to cell and embryos during processing and enhances their further development.

### CROSS-REFERENCE TO RELATED APPLICATION

This application is a continuation-in-part (CIP) of International Application No. PCT/CN2023/109867, filed on Jul. 28, 2023, the entire disclosure of which is incorporated by reference herein.

### FIELD

The disclosure relates to a specimen container for reproductive medicine, and more particularly to a specimen container for collecting and processing semen, ova, embryos and cell clusters.

### BACKGROUND

Recently, much attention has been paid to the issue of infertility. In reproductive medicine, it is generally recommended to adopt to in vitro fertilization (IVF) or artificial fertilization (so-called test tube baby) to solve the problem of infertility. During IVF processes, healthy sperm are selected to be combined with an ovum. Sperm motility is evaluated after the sperm separation and selection procedures. The common sperm collection methods are swim-up and density gradient separation, in which both method procedures require the centrifugal separation. Conventionally, further selection of sperms for natural insemination or ICSI (Intracytoplasmic Sperm Injection) fertilization is manually prepared. The manual selection is conducted under the microscope regarding to the reactivity, motility, and morphology of gametes and embryos. Also, a further manipulation with gametes and embryos includes activation, immobilization, hatching, and biopsy. Thus, the collection and selection of gametes and embryos are time consuming, and also cause damage to the sperms, oocytes or embryos and hence reduce the success rate of conception.

Moreover, the sperm is manually washed in a repeated manner to remove proteins, secretions, cells, bacteria and substances which might interfere with fertilization, and the ovum is washed to remove granulosa cells, proteoglycans, proteins, hyaluronic acid and other secretions around the ovum which affect the chances of conception and hinder the next step of treatment. In the cryopreservation and subsequently thawing procedures of oocytes and embryos, the procedures requiring strictly timely transferring and immersion in different media are easily affected by human and environmental errors. Also, this manner of washing or stripping might cause damage to the ovum, embryos or cell cluster, and hence may lead to reduced pregnancy rates or subsequent cell developments. In addition, the preparation of sperm or the treatment of ovum and embryos in the IVF process is generally conducted in an environment that may be affected by human error. Also, special culture environment and atmosphere compositions for the embryos or cells may be ruptured in these manual processing. Therefore, an automated equipment for use in the field of reproductive medicine and bioengineering culture may facilitate and standardize these procedures.

### SUMMARY

Therefore, an object of the disclosure is to provide a specimen container for reproductive medicine that can alleviate at least one of the drawbacks of the prior art.

According to an aspect of the disclosure, there is provided a specimen container for reproductive medicine according to claim 1.

According to an aspect of the disclosure, there is provided a specimen container for reproductive medicine according to claim 7.

According to an aspect of the disclosure, there is provided a specimen container for reproductive medicine according to claim 9.

According to an aspect of the disclosure, there is provided a specimen container for reproductive medicine according to claim 14.

According to an aspect of the disclosure, there is provided a specimen container for reproductive medicine according to claim 21.

According to an aspect of the disclosure, there is provided a specimen container for reproductive medicine according to claim 34.

### BRIEF DESCRIPTION OF THE DRAWINGS

Other features and advantages of the disclosure will become apparent in the following detailed description of the embodiments with reference to the accompanying drawings. It is noted that various features may not be drawn to scale.

FIG. 1 is an exploded perspective view illustrating a first embodiment of a specimen container according to the disclosure.

FIG. 2 is a sectional view of the first embodiment.

FIG. 3 is a sectional view of the first embodiment in a modified form.

FIG. 4 is a sectional view of the first embodiment in another modified form.

FIG. 5 is an exploded perspective view of the first embodiment in still another modified form.

FIG. 6 is an exploded perspective view illustrating a second embodiment of a specimen container according to the disclosure.

FIG. 7 is an exploded perspective view illustrating a third embodiment of a specimen container according to the disclosure.

FIG. 8 is a fragmentary exploded perspective view of the third embodiment.

FIG. 9 is a cross-sectional view of the third embodiment.

FIG. 10 is a sectional view taken along line X-X of FIG. 9.

FIG. 11 is an exploded perspective view illustrating a fourth embodiment of a specimen container according to the disclosure.

FIG. 12 is a top view of the fourth embodiment.

FIG. 13 is a top view of the fourth embodiment in a modified form.

FIG. 14 is a cross-sectional view of the fourth embodiment in another modified form.

FIG. 15 is a sectional view taken along line XV-XV of FIG. 14.

FIG. 16 is an exploded perspective view illustrating a fifth embodiment of a specimen container according to the disclosure.

FIG. 17 is a fragmentary exploded perspective view of the fifth embodiment.

FIG. 18 is a cross-sectional view of the fifth embodiment in a modified form.

FIG. 19 is a cross-sectional view of the fifth embodiment in another modified form.

FIG. 20 is a cross-sectional view of the fifth embodiment in still another modified form.

FIG. 21 is a fragmentary exploded perspective view of the fifth embodiment, illustrating a separating piece in a modified form.

FIG. 22 is a sectional view of the fifth embodiment in FIG. 21.

FIG. 23 is a cross-sectional view of the fifth embodiment in FIG. 21.

FIG. 24 is a cross-sectional view of the fifth embodiment, illustrating communicating holes of the separating piece in a modified form.

FIG. 25 is a fragmentary exploded perspective view illustrating a sixth embodiment of a specimen container according to the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

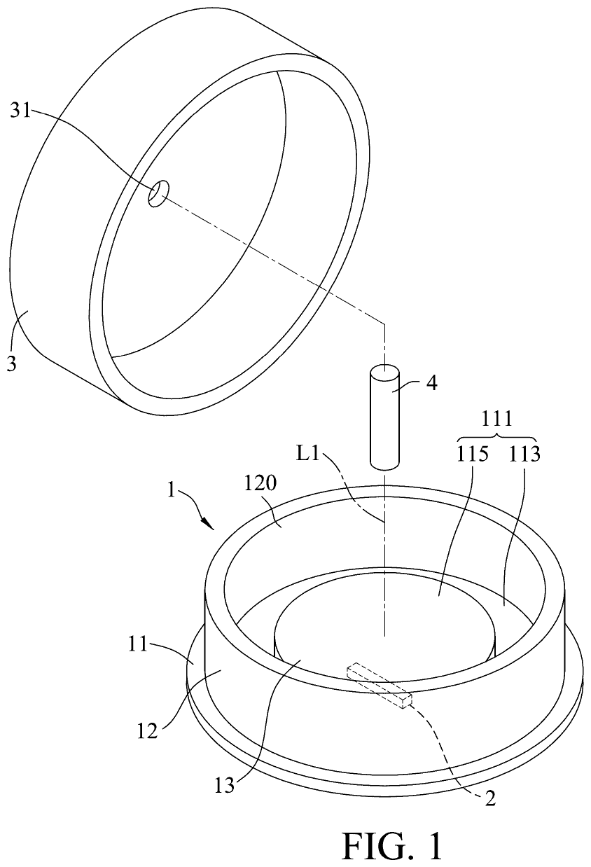

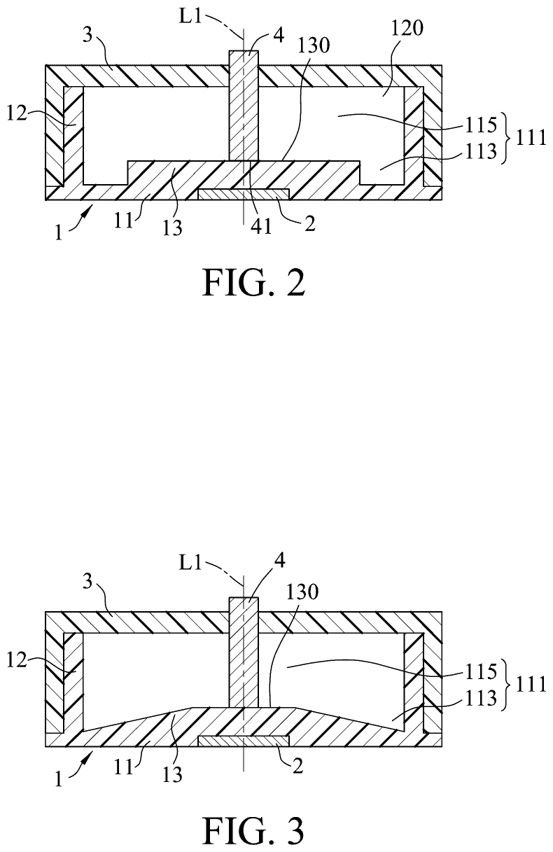

Referring to FIGS. 1 and 2, a first embodiment of a specimen container is adapted to collect a semen specimen and to select motile sperms from the specimen. The semen specimen is made from mixing semen with a buffer solution. The specimen container includes a container body 1, a rotated element 2 disposed on a bottom portion of the container body 1, a cap 3 removably mounted on the container body 1, and a barrier element 4 removably mounted on the cap 3 and inserted into the container body 1.

The container body 1 includes a base wall 11, a surrounding wall 12 which extends upwardly from the base wall 11 and which surrounds a central axis (L1) to cooperate with the base wall 11 to define a specimen receiving space 111 with an upwardly facing opening 120, and a protrusion 13 which projects upwardly from the base wall 11 to terminate at an upper surface 130 and which is formed in the specimen receiving space 111. The protrusion 13 is in the form of a circular disc with the flat upper surface 130 and is coaxial with the central axis (L1) such that the specimen receiving space 111 has an outer surrounding region 113 which is bordered between the protrusion 13 and an inner peripheral surface of the surrounding wall 12, and a central region 115 which is formed on and above the upper surface 130.

The rotated element 2 is a magnetically driven element which is operated by an external magnetic force to be rotated about the central axis (L1) so as to rotate the container body 1 about the central axis (L1). The rotating operation of the rotated element 2 may be an intermittent or continuous rotating operation. Alternatively, the rotated element 2 may have a structure coupled with and driven by a motor mechanism.

The cap 3 is removably mounted on the surrounding wall 12 of the container body 1 to cover the opening 120 of the specimen receiving space 111. The cap 3 has a through hole 31 extending therethrough along the central axis (L1) to communicate the specimen receiving space 111 with ambient air.

The barrier element 4 is removably extended through the through hole 31 along the central axis (L1), and has a lower end which abuts against the upper surface 130 of the protrusion 13 when the cap 3 is mounted on the container body 1.

In the first embodiment, the specimen receiving space 111 has a diameter ranging from 10 mm to 30 mm, and the outer surrounding region 113 has a diameter ranging from 1 mm to 3 mm.

In use, the cap 3 and the barrier element 4 are firstly removed from the container body 1, a specimen of about 1 to 3 ml is dropped into the specimen receiving space 111 of the container body 1, enough to submerge the protrusion 13. Subsequently, the cap 3 is mounted on the container body 1, and the barrier element 4 is extended through the cap 3 to have the lower end thereof abut against the upper surface 130 of the protrusion 13. Thus the specimen in the specimen receiving space 111 is moved outwardly away from the center region 115 through which the central axis (L1) passes at which the weakest centrifugal force is derived so that the specimen can be subjected to the centrifugal force more effectively.

Next, the specimen container is loaded on a centrifugal device (not shown) to drive the rotated element 2 to rotate the container body 1, the cap 3 and the protrusion 13 about the central axis (L1) so as to perform a centrifugal process of the specimen in the specimen receiving space 111. In the centrifugal process, the sperm, protein and secretions in the specimen are separated and moved in the outer surrounding region 113. After the centrifugal process is completed, the specimen container is rested for a predetermined time. During the resting, the motile sperms gradually spread and swim in a variety of directions, and the most motile sperms are able to overcome the physical obstacles set by the protrusion 13 and move to the central region 115.

Finally, without removal of the cap 3 from the container body 1, the barrier element 4 is withdrawn from the cap 3 so that the most motile sperms can be extracted from the central region 115 to complete the sperm selection and separation.

With reference to FIG. 3, in a modified form of the protrusion 13, the protrusion 13 has a frusto-conical cross-section to have the upper surface 130 gradually raised from a peripheral portion to the central axis (L1).

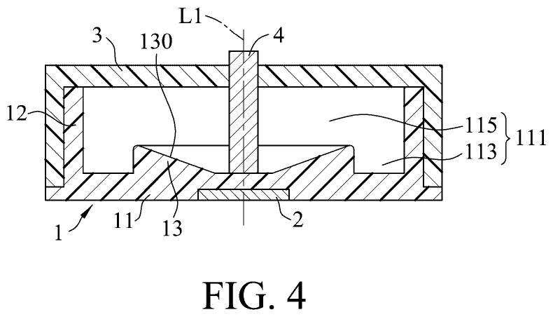

With reference to FIG. 4, in another modified form of the protrusion 13, the protrusion 13 is concaved to have the upper surface 130 inclined and lowered from a peripheral portion to the central axis (L1).

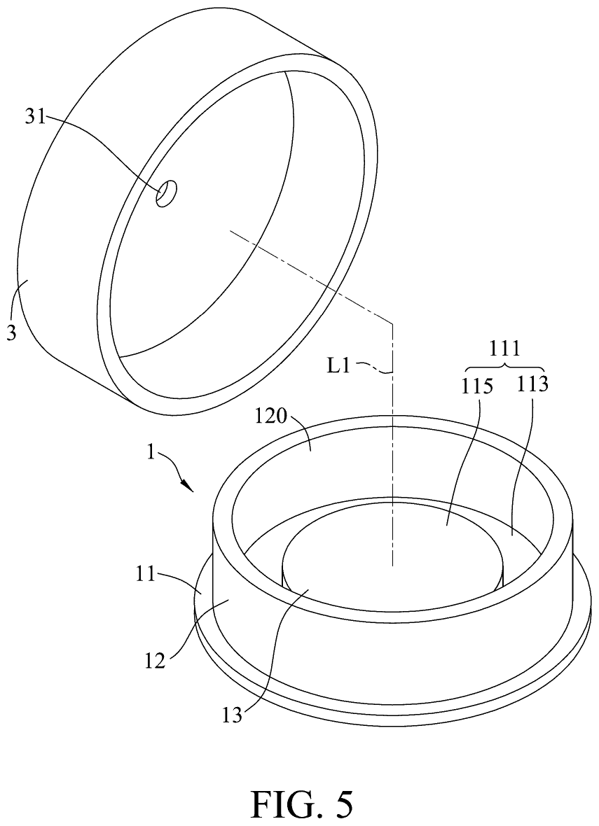

With reference to FIG. 5, also, the specimen container of the first embodiment may only include the container body 1 and the cap 3. In use, the cap 3 is removed from the container body 1. A semen sample is dropped into the outer surrounding region 113 and a buffer solution is dropped into the specimen receiving space 111, enough to submerge the protrusion 13. The cap 3 is mounted on the container body 3 to cover the opening 120 of the specimen receiving space 111. Subsequently, the specimen container is rested. The motile sperms gradually spread and swim from the outer surrounding region 113 in a variety of directions, and the most motile sperms are able to overcome the physical obstacles set by the protrusion 13 and move to the central region 115. Finally, without removal of the cap 3 from the container body 1, the most motile sperms can be extracted from the central region 115 through the through hole 31 to complete the sperm selection.

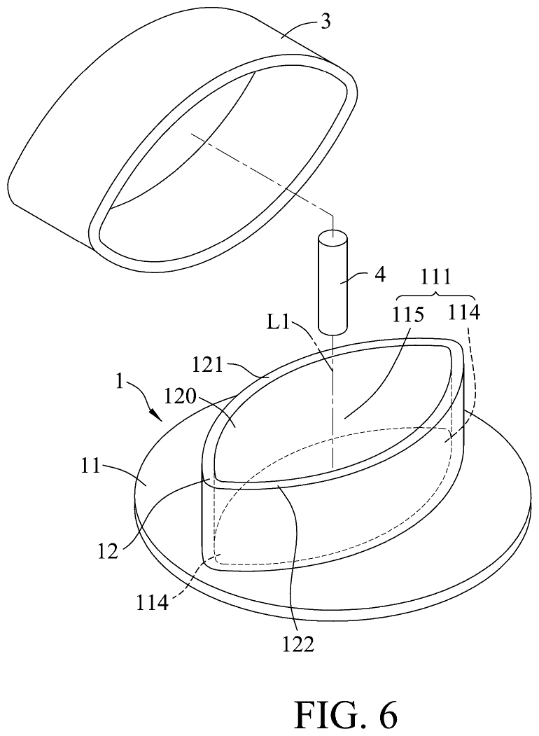

With reference to FIG. 6, in a second embodiment of the specimen container, the protrusion is dispensed with, and the surrounding wall 12 of the container body 1 has an almond shaped cross-section. Specifically, the surrounding wall 12 includes a first arcuate wall half 121 which extends arcuately to have two first wall ends, and a second arcuate wall 122 which extends arcuately to have two second wall ends that are respectively connected with the first wall ends of the first arcuate wall half 121 such that the specimen receiving space 111 has two separating pointed regions 114 which are bordered by the first and second wall ends, and a central region 115 which is interposed between the separating pointed regions 114 and through which the central axis (L1) passes.

In use, the separating pointed regions 114 and the central region 115 will be subjected to different centrifugal forces (according to distance from the central axis (L1), the sperm are induced to move to and may be collected from the separating pointed regions 114 after the centrifugal process. The most motile sperm are able to move from the separating pointed regions 114 to the central region 115 so as to be extracted from the central region 115 to complete the sperm selection and separation. It is noted that the specimen container of the disclosure can be adapted to select and separate a variety of mobile objects, and is useful not only for sperm selection.

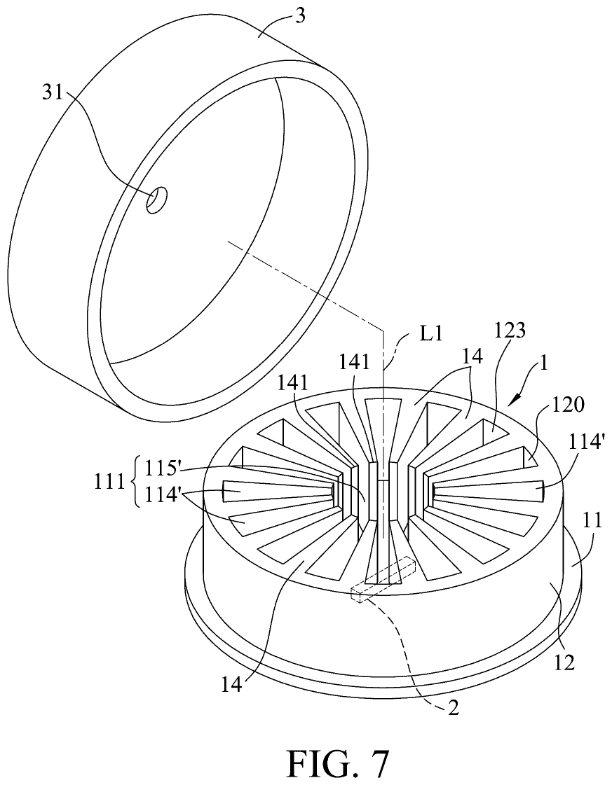

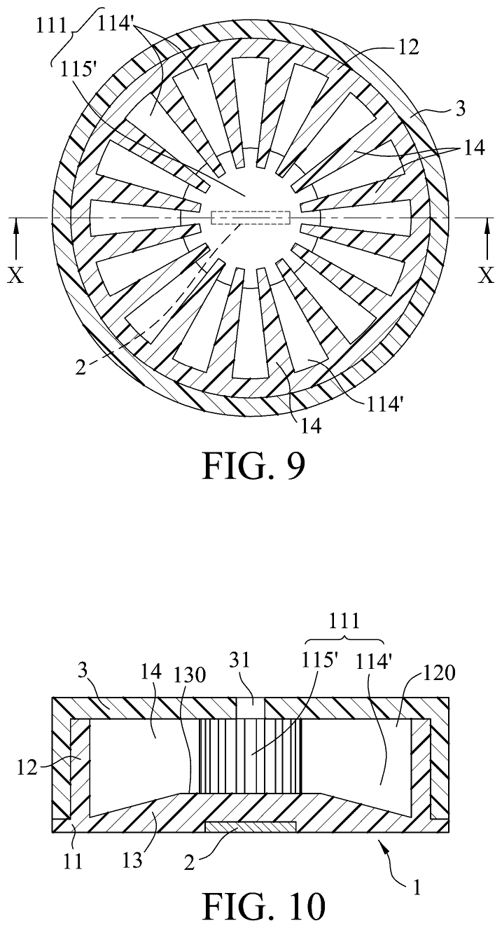

With reference to FIGS. 7, 8 and 9, in a third embodiment of the specimen container, the barrier element is dispensed with, and the container body 1 has a different structure from the previous embodiments.

The container body 1 further includes a plurality of partition walls 14 (16 partition walls 14 in this embodiment), each of which extends radially from an inner peripheral surface 123 of the surrounding wall 12 toward the central axis (L1) to terminate at an end edge 141. The partition walls 14 extend upwardly from the base wall 11 and are angularly spaced apart from one another to divide the specimen receiving space 111 into a plurality of separating regions (114′) (16 separating regions in this embodiment) between the end edges 141 and the inner peripheral surface 123, and a central region (115′) which is communicated with the separating regions (114′) and which is bordered by the end edges 141. Through the partition walls 14, sperm that are able to travel in a straight line can be selected by extracting the specimen from the central region (115′).

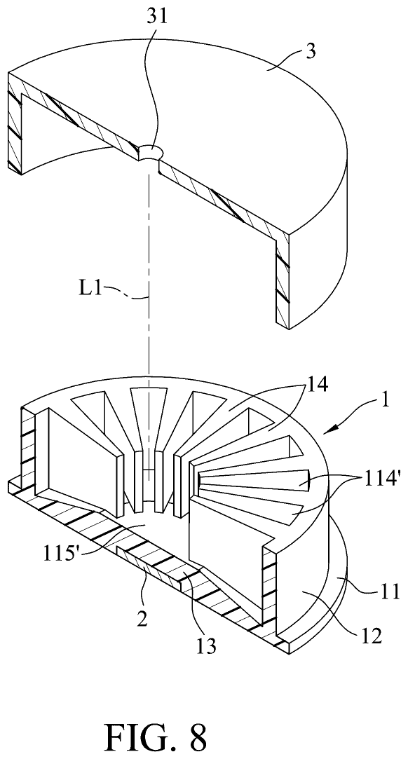

With reference to FIGS. 8, 9 and 10, in this embodiment, the protrusion 13 has a frusto-conical cross-section to have the upper surface 130 gradually raised from a peripheral portion to the central axis (L1). The protrusion 13 may be a variety of configurations. In use, through vertical obstacles set by the protrusion 13 and horizontal obstacles set by the partition walls 14, sperms that are able to move from the separating regions (114′) to the central region (115′) are selected.

In a modified form, the protrusion 13 is dispensed with, and only through the horizontal obstacles set by the partition walls 14 are motile sperms selected.

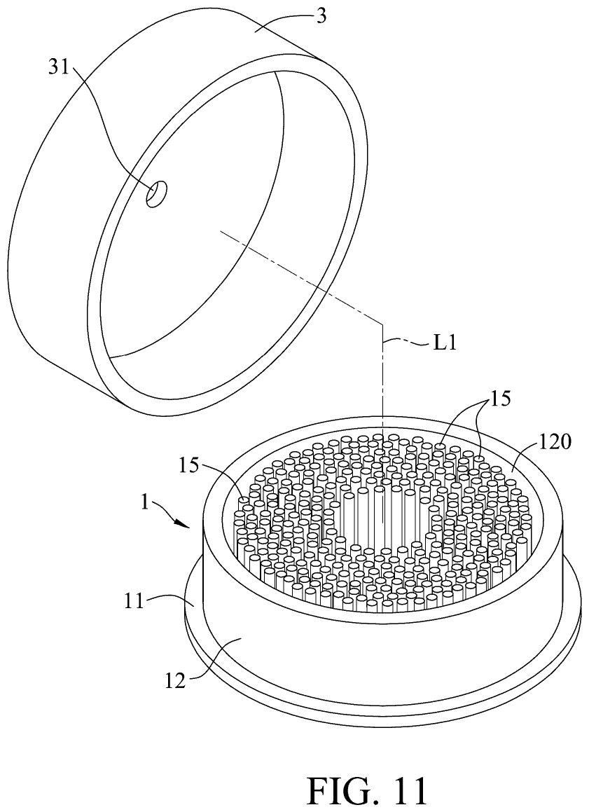

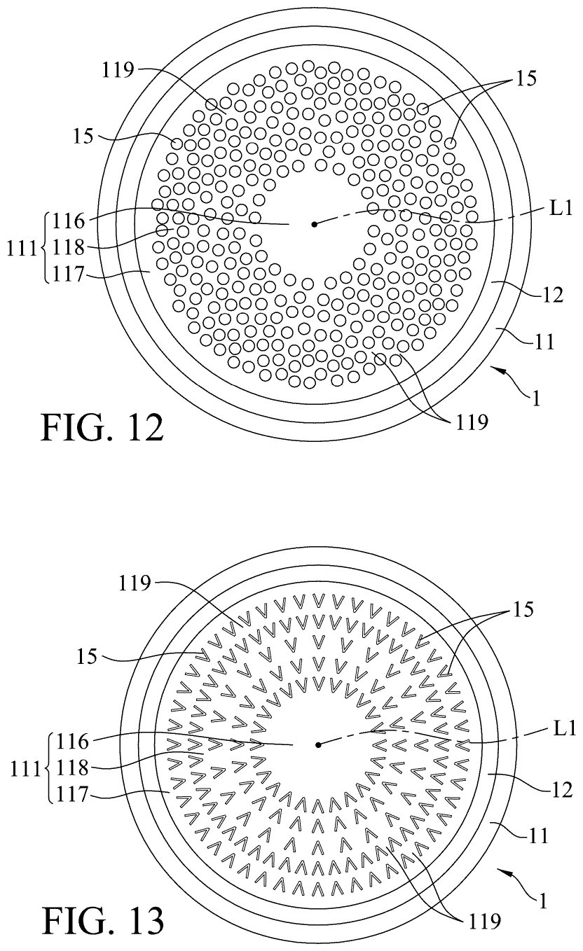

With reference to FIGS. 11 and 12, in a fourth embodiment, the container body 1 has a different structure from the previous embodiments. The container body 1 further includes a plurality of barrier poles 15 which extend upwardly from the base wall 11 and which are spaced apart from one another. The barrier poles 15 are arranged in and divide the specimen receiving space 111 into a central region 116 which is defined inwardly of the barrier poles 15, a surrounding region 117 which is defined outwardly of the barrier poles 15 and which surrounds the central region 116, and a gapped region 118 which is interposed between the central region 116 and the surrounding region 117 and in which the barrier poles 15 are disposed.

Each of the barrier poles 15 has a circular cross-section, and the gapped region 118 has a plurality of non-straight linear gaps 119 defined by the barrier poles 15 and communicating the central region 116 with the surrounding region 117.

After the centrifugal process of the sperm specimen, motile sperms travel through the gapped region 118 in which the barrier poles 15 are disposed, and are able to be selected by extracting the sperms from the central region 116.

With reference to FIG. 13, in a modified form of the barrier poles 15, each of the barrier poles 15 has a V-shaped cross-section with a notch opened toward the surrounding region 117. Sperms are required to travel from the surrounding region 117 and bypass the opened notches of the barrier poles 15 in order to reach the central region 116. Hence, motile sperms may be selected from the central region 116.

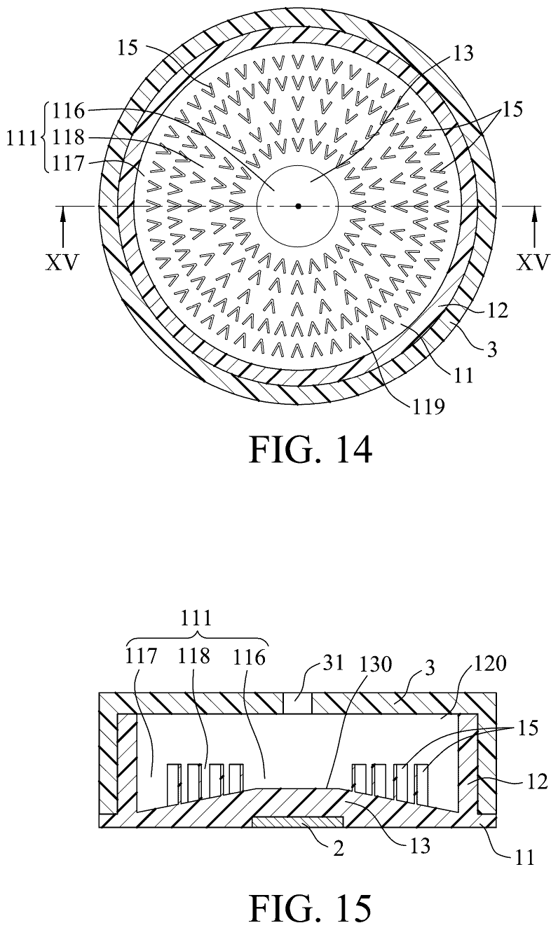

With reference to FIGS. 14 and 15, in a modified form of the fourth embodiment, the container body 1 includes the protrusion 13. For example, the protrusion 13 may be similar to that shown in FIG. 3. The barrier poles 15 project from the upper surface 130 of the protrusion 13. In use, after subjugating the sperm specimen to the centrifugal process, through vertical obstacles set by the protrusion 13 and physical obstacles set by the barrier poles 15, sperms that are able to move from the surrounding region 117 to the central region 116 may be selected by extracting the sperms from the central region 116.

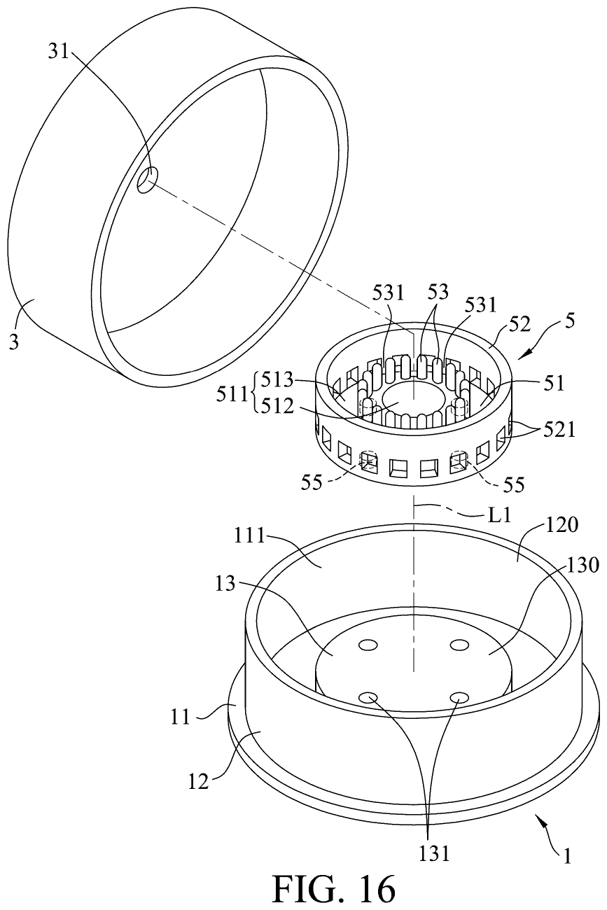

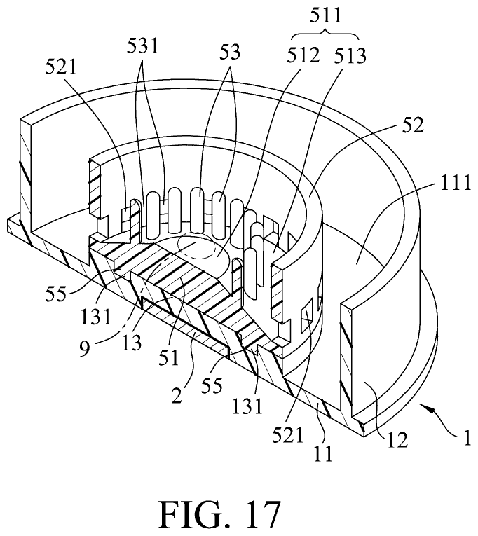

With reference to FIGS. 16 and 17, a fifth embodiment of the specimen container according to the disclosure is adapted to wash and separate ovum from a follicular fluid specimen. The specimen is made from mixing ovum, proteins, and secretions with a buffer solution. The container body 1 has a different structure from the previous embodiments, and the specimen container of this embodiment further includes a separating piece 5 which is removably mounted on the container body 1.

The container body 1 includes a base wall 11, a surrounding wall 12 and a protrusion 13 which are similar to those in the previous embodiments. The upper surface 130 of the protrusion 13 has a plurality of first engaging portions 131.

The separating piece 5 includes a bottom wall 51 which is removably retained on the upper surface 130 of the protrusion 13, a peripheral wall 52 which extends upwardly from the bottom wall 51 and which surrounds the central axis (L1) to cooperate with the bottom wall 51 to define a separating space 511, a plurality of barrier poles 53 which extend upwardly from the bottom wall 51 and which are angularly spaced apart from one another along the peripheral wall 52, and a plurality of second engaging portions 55 which are disposed on the bottom wall 51 and which are respectively and removably engaged with the first engaging portions 131 to removably retain the separating piece 5 on the container body 1.

In this embodiment, the bottom wall 51 has a frusto-conical cross-section and has an upper wall surface which is gradually raised from a peripheral portion to a flat central portion through which the central axis (L1) passes. Alternatively, the bottom wall 51 may be in the form of a circular layer with a raised portion or without any raised portion.

The peripheral wall 52 has a plurality of communicating holes 521 which extend radially therethrough to communicate the separating space 511 with the specimen receiving space 111.

Each of the barrier poles 53 has an upper pole end which is of an arcuate shape, and the barrier poles 53 are arranged to surround the central axis (L1) to be flush with one another. The barrier poles 53 divide the separating space 511 into a central compartment 512 which is defined inwardly of the barrier poles 53, a surrounding compartment 513 which is defined outwardly of the barrier poles 53 and which is communicated with the communicating holes 521, and a plurality of slits 531 each of which is defined between two adjacent ones of the barrier poles 53 and which communicate the central compartment 512 with the surrounding compartment 513. In this embodiment, the barrier poles 53 are lower than the peripheral wall 52 by 300 μm. The width of the slits 531 ranges from 30 μm to 50 μm for selecting ovum and human embryos.

The second engaging portions 55 project downwardly from the bottom wall 51 to be engaged with the first engaging portions 131 that are recessed from the upper surface 130 of the protrusion 13. Alternatively, the first and second engaging portions 131, 55 may have a magnetically attractive engagement with each other so as to removably retain the separating piece 5 on the container body 1.

In use, a specimen is dropped into the central compartment 512 through the through hole 31. The specimen flows from the central compartment 512 through the surrounding compartment 513 and into the specimen receiving space 111 until the height of the specimen reaches the height of the barrier poles 53. After conducting the rotational centrifugal process on the sperm specimen, an ovum 9 in the specimen will be blocked by the barrier poles 53 due to it having a larger diameter than the width of the slits 531, and is kept in the central compartment 512 while the granulosa cells attached to the periphery of the ovum 9, protein and secretions will be flung by the centrifugal force from the central compartment 512 to the surrounding compartment 513 through the slits 531. They are then discharged into the specimen receiving space 111 through the through holes 521. Thus, the washing and separating process of the ovum 9 is performed.

Subsequently, the cap 3 is removed from the container body 1, and the second engaging portions 55 of the separating piece 5 are removed from the first engaging portions 131 to remove the separating piece 5 from the container body 1 so that a subsequent process may be carried out without the need to replace the container.

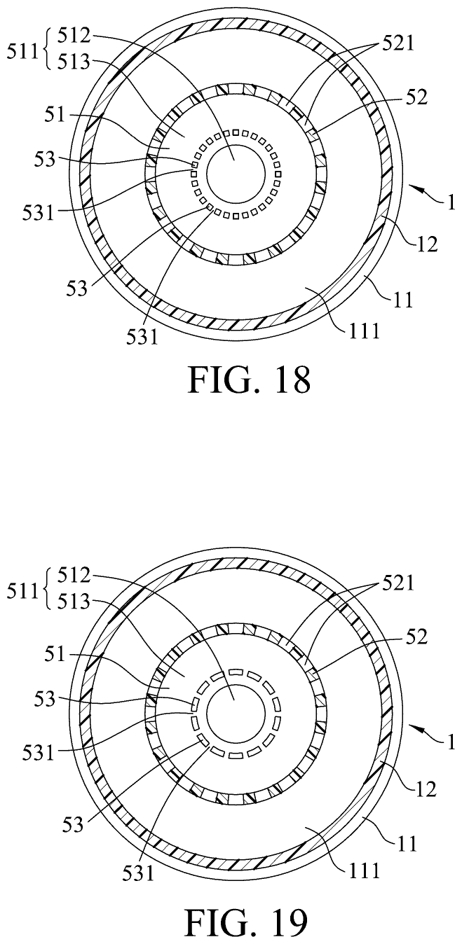

With reference to FIGS. 18 and 19, in a modified form of the fifth embodiment, each barrier pole 53 has a rectangular cross-section.

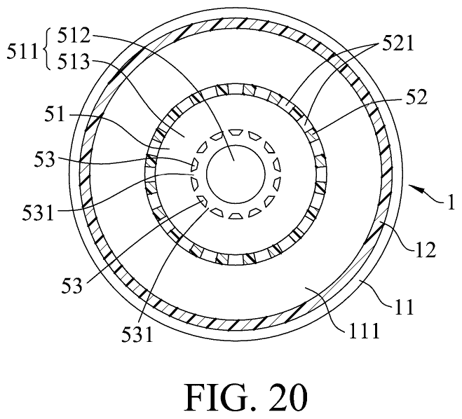

With reference to FIG. 20, in another modified form of the fifth embodiment, each slit 531 has a width which is measured between two adjacent ones of the barrier poles 53 and which gradually decreases from the central compartment 512 to the surrounding compartment 513.

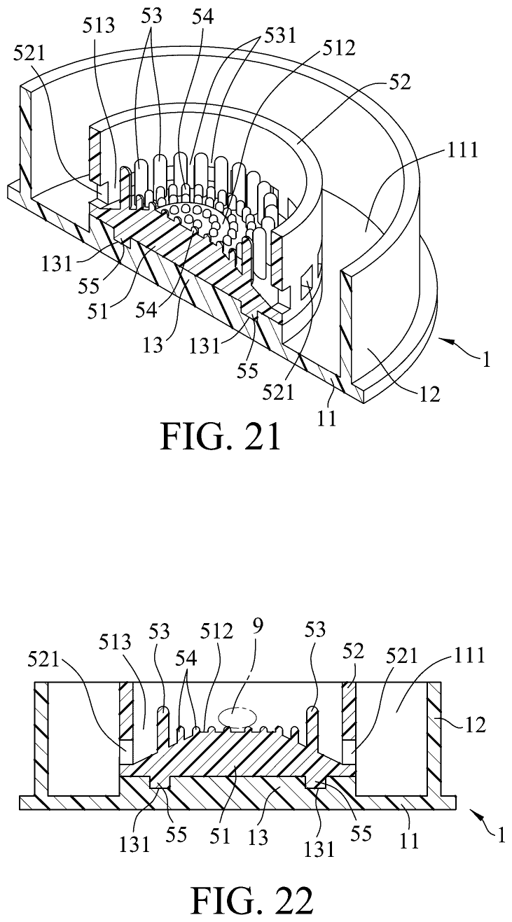

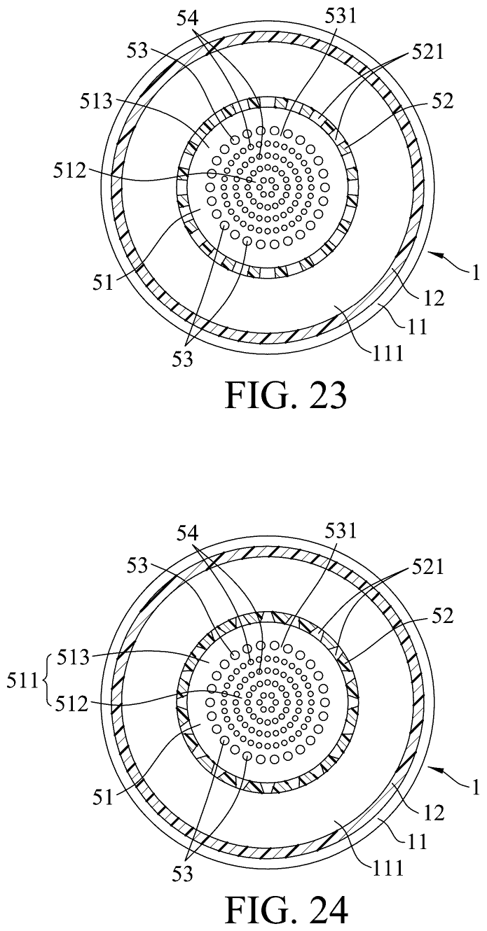

With reference to FIGS. 21, 22 and 23, in still another modified form of the fifth embodiment, the separating piece 5 further includes a plurality of separating studs 54 which are formed on the bottom wall 51 in the separating space 511 and arranged inwardly of the barrier poles 53 so as to brace the ovum 9 when a specimen is dropped into the central compartment 512. Each of the separating studs 54 has an upper stud end which is of an arcuate shape.

After the rotational centrifugal process of the sperm specimen, impurities around the ovum 9 may be separated from the ovum 9 by friction with the separating studs 54, and the ovum 9 will be blocked at a height position between the barrier poles 53 and the separating studs 54 due to the height of the barrier poles 53 being higher than that of the separating studs 54, or the ovum 9 may fall into the surrounding compartment 513 but will be blocked by the peripheral wall 52. The washing and separating process of the ovum 9 may also be performed.

With reference to FIG. 24, in other modified form of the fifth embodiment, each of the communicating holes 521 has an inner diameter which gradually decreases from the separating space 511 to the specimen receiving space 111.

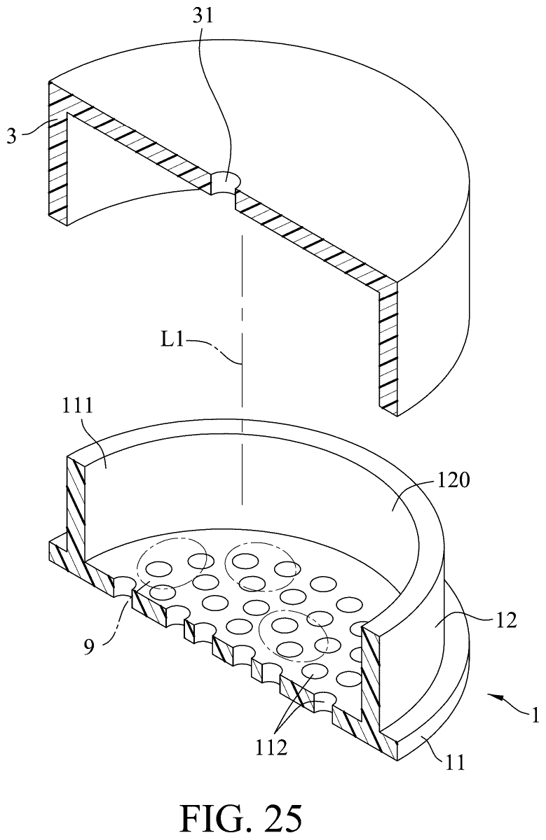

With reference to FIG. 25, in a sixth embodiment of the specimen container, the rotated element is dispensed with, and the container body 1 has a different structure from the previous embodiments.

In this embodiment, the container body 1 only includes a base wall 11 and a surrounding wall 12. The base wall 11 has a plurality of penetrating holes 112 which extend therethrough in a direction of the central axis (L1) to be communicated with the specimen receiving space 111.

In the specimen container of the sixth embodiment, each penetrating hole 112 has a diameter ranging from 50 μm to 80 μm which is adapted for processing ovum and human embryos, or a diameter ranging from 20 μm to 40 μm to which is adapted for processing human primordial follicle or mouse ovum and embryos.

In use, a specimen is dropped into the specimen receiving space 111 through the through hole 31 of the cap 3. The specimen container is then loaded on an oscillation device (not shown) to oscillate the specimen container so as to separate the ovum 9 from the specimen. The ovum 9 is blocked and kept in the specimen receiving space 111 due to the ovum 9 having a diameter that is larger than that of the penetrating holes 112, and other impurities may pass through and be discharged from the penetrating holes 112. Subsequently, the specimen container carries the clean ovum 9 and a subsequent process may be carried out without the need to replace the container for preventing undesired damage to the ovum 9.

As illustrated, the structure of the container body 1 is designed to be suitable for selecting semen or processing ovum which can reduce damage to sperm and ovum during processing.

Specifically, with the rotated element 2 operated to rotate the container body 1 about the central axis (L1), the specimen container can perform a centrifugal process on the specimen. The barrier element 4 is removably disposed along the central axis (L1) such that the specimen can be subjected to the centrifugal force more effectively. Also, the barrier element 4 is removed from the cap 3 to expose the through hole 31 to facilitate extracting sperms in the specimen from the central region 115 through the through hole 31. The separating piece 5 is removably mounted on the protrusion 13 for easier processing of the ovum 9 and simplifies the subsequent process. With the outer surrounding region 113, the separating pointed regions 114 and the separating regions (114′), sperms and other impurities can be collected via application of the centrifugal force. With the protrusion 13, vertical obstacles can be set for selecting sperms motile in an up-down direction. With the partition walls 14, horizontal obstacles can be set for selecting sperms motile in a horizontally forward direction. With the barrier poles 15, physical obstacles can be set for selecting motile sperms. That is, with the obstacles set by the protrusion 13, the partition walls 14 and the barrier poles 15, the difficulty of sperms swimming back to the central region 115 from the outer surrounding region 113 is increased, which is used as a basis for selecting motile sperms. Through the rotational centrifugal or oscillating process, and with the barrier poles 53, the specimen container can separate out, or disperse impurities from an ovum 9 for selection. Moreover, with the separating piece 5, the specimen container is adapted to clean and process the ovum 9, and the cleaned ovum 9 is kept in the separating piece 5 for the subsequent process without replacement of the container, which reduces damage to the ovum. Therefore, the present invention is an automated multifunctional device for application in reproductive medicine.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A specimen container for reproductive medicine, comprising:
a container body including a base wall, and a surrounding wall which extends upwardly from said base wall and which surrounds a central axis to cooperate with said base wall to define a specimen receiving space with an upwardly facing opening, said specimen receiving space having a central region through which the central axis passes.

2. The specimen container of claim 1, wherein said container body further includes a protrusion which projects upwardly from said base wall to terminate at an upper surface, said protrusion being formed in said specimen receiving space and defining said central region on and above said upper surface, said specimen receiving space further having an outer surrounding region which is bordered between said protrusion and an inner peripheral surface of said surrounding wall.

3. The specimen container of claim 2, further comprising
a rotated element which is disposed on said base wall and which is a magnetically driven element that is operated by an external magnetic force to be rotated about the central axis;
a cap which is removably mounted on said container body to cover said opening of said specimen receiving space, said cap having a through hole extending therethrough along the central axis to communicate said specimen receiving space with ambient air; and
a barrier element which is removably extended through said through hole along the central axis and which has a lower end that abuts against said upper surface of said protrusion when said cap is mounted on said container body.

4. The specimen container of claim 1, wherein
said surrounding wall includes a first arcuate wall half which extends arcuately to have two first wall ends, and a second arcuate wall which extends arcuately to have two second wall ends that are respectively connected with said first wall ends of said first arcuate wall half, said specimen receiving space further having two separating pointed regions which are bordered by said first and second wall ends, said central region being interposed between said separating pointed regions.

5. The specimen container of claim 4, further comprising a cap which is removably mounted on said container body to cover said opening of said specimen receiving space.

6. The specimen container of claim 1, wherein
said container body further includes a plurality of partition walls each of which extends radially from an inner peripheral surface of said surrounding wall and toward the central axis to terminate at an end edge, said partition walls extending upwardly from said base wall and being angularly spaced apart from one another to divide said specimen receiving space into a plurality of separating regions between said end edges and said inner peripheral surface, and said central region which is communicated with said separating regions and which is bordered by said end edges.

7. The specimen container of claim 6, further comprising a rotated element which is disposed on said base wall and which is a magnetically driven element that is operated by an external magnetic force to be rotated about the central axis, and a cap which is removably mounted on said container body to cover said opening of said specimen receiving space, said cap having a through hole extending therethrough to communicate said specimen receiving space with ambient air.

8. The specimen container of claim 1, wherein
said container body further includes a plurality of barrier poles which extend upwardly from said base wall and which are spaced apart from one another, said barrier poles being arranged in and dividing said specimen receiving space into said central region which is defined inwardly of said barrier poles, a surrounding region which is defined outwardly of said barrier poles and which surrounds said central region, and a gapped region which is interposed between said central region and said surrounding region and in which said barrier poles are disposed.

9. The specimen container of claim 8, wherein each of said barrier poles has a circular cross-section, and said gapped region has a plurality of non-straight linear gaps defined by said barrier poles and communicating said central region with said surrounding region.

10. The specimen container of claim 8, wherein each of said barrier poles has a V-shaped cross-section with a notch opened toward said surrounding region, and said gapped region has a plurality of non-straight linear gaps defined by said barrier poles and communicating said central region with said surrounding region.

11. The specimen container of claim 8, further comprising a rotated element which is disposed on said base wall and which is a magnetically driven element that is operated by an external magnetic force to be rotated about the central axis, and a cap which is removably mounted on said container body to cover said opening of said specimen receiving space, said cap having a through hole extending therethrough to communicate said specimen receiving space with ambient air.

12. A specimen container for reproductive medicine, comprising:
a container body including a base wall, a surrounding wall which extends upwardly from said base wall and which surrounds a central axis to cooperate with said base wall to define a specimen receiving space with an upwardly facing opening, and a protrusion which projects upwardly from said base wall to terminate at an upper surface and which is formed in said specimen receiving space; and
a separating piece including a bottom wall which is removably retained on said upper surface of said protrusion, a peripheral wall which extends upwardly from said bottom wall and which surrounds the central axis to cooperate with said bottom wall to define a separating space, and a plurality of barrier poles which extend upwardly from said bottom wall and which are angularly spaced apart from one another along said peripheral wall, said peripheral wall having a plurality of communicating holes which extend radially therethrough to communicate said separating space with said specimen receiving space.

13. The specimen container of claim 12, wherein said bottom wall has a frusto-conical cross-section and has an upper wall surface which is gradually raised from a peripheral portion to a flat central portion through which the central axis passes.

14. The specimen container of claim 12, wherein said barrier poles are arranged to surround the central axis and divide said separating space into a central compartment which is defined inwardly of said barrier poles, a surrounding compartment which is defined outwardly of said barrier poles and which is communicated with said communicating holes, and a plurality of slits each of which is defined between two adjacent ones of said barrier poles and which communicate said central compartment with said surrounding compartment.

15. The specimen container of claim 14, wherein said separating piece further includes a plurality of separating studs which are formed on said bottom wall in said separating space and arranged inwardly of said barrier poles, each of said barrier poles having an upper pole end which is of an arcuate shape, each of said separating studs having an upper stud end which is of an arcuate shape, said barrier poles extending flush with one another, and having a height higher than that of said separating studs.

16. The specimen container of claim 12, wherein said upper surface of said protrusion has a plurality of first engaging portions, said separating piece further including a plurality of second engaging portions which are disposed on said bottom wall and which are respectively and removably engaged with said first engaging portions to removably retain said separating piece on said container body.

17. The specimen container of claim 12, wherein each of said communicating holes has an inner diameter which gradually decreases from said separating space to said specimen receiving space.

18. The specimen container of claim 12, further comprising a rotated element which is disposed on said base wall and which is of a magnetically driven element that is operated by an external magnetic force to be rotated about the central axis, and a cap which is removably mounted on said container body to cover said opening of said specimen receiving space, said cap having a through hole extending therethrough to communicate said specimen receiving space with ambient air.

19. A specimen container for reproductive medicine, comprising:
a container body including a base wall and a surrounding wall which extends upwardly from said base wall and which surrounds a central axis to cooperate with said base wall to define a specimen receiving space with an upwardly facing opening, said base wall having a plurality of penetrating holes which extend therethrough in a direction of the central axis to be communicated with said specimen receiving space.

20. The specimen container of claim 19, further comprising a cap which is removably mounted on said container body to cover said opening of said specimen receiving space, said cap having a through hole extending therethrough to communicate said specimen receiving space with ambient air.
